(19) United States
(12) Patent Application Publication
(10) Pub. No.: US 2012/0058158 A1
(43) Pub. Date: Mar. 8, 2012

(54) Sublingual Pharmaceutical Composition Comprising a Neutral Oil

(75) Inventor: Clive Booles, Norfolk (GB)

(73) Assignee: LONDONPHARMA LTD., Norwich (GB)

(21) Appl. No.: 13/265,825

(22) PCT Filed: Apr. 23, 2010
(86) PCT No.: PCT/GB2010/050671

§ 371 Date: Nov. 17, 2011

(30) Foreign Application Priority Data
Apr. 23, 2009 (GB) 0906977.4
Apr. 23, 2009 (GB) PCT/GB2009/050416

(51) Int. Cl. A61K 9/08; A61P 25/04; A61K 47/44; A61K 47/46; A61K 47/10

(52) U.S. Cl. 424/400; 514/783; 514/772

(57) ABSTRACT

The invention provides pharmaceutical compositions for the sublingual delivery of medicaments comprising a neutral oil and a medicament soluble in said oil, providing that said medicament is not nitroglycerine. The invention also provides delivery devices adapted for sublingual delivery of such compositions.

### FIELD OF THE INVENTION

The invention relates to improved methods of delivery for medicaments, and to devices for drug delivery.

### BACKGROUND

The development of drug delivery routes remains an important element in the progress of the pharmaceutical sciences. Once an active compound has been identified, the design of delivery mechanisms must overcome challenges of transporting the medicament to the required site of action in the body whilst addressing issues including shelf stability, bioavailability, toxicity, and patient compliance. All of these challenges must be overcome to achieve the desired therapeutic effect. Amongst the drug delivery options, oral administration is by far the most common route, with other options including injection, topical, inhalation and transmucosal administration.

The oral delivery route faces perhaps the most challenging route for a pharmaceutical to reach the final site of action: The composition must survive the acidic and enzymatically-active environment of the stomach; if not absorbed in the stomach, the medicament must survive the action of bile salts and further intestinal and bacterial enzymatic action within the intestinal tract, be able to cross from the lumen of the gut to the intestinal wall for absorption, and then survive the degradation processes of the liver following transport by the hepatic portal system, often resulting in poor availability due to the first pass effect. Furthermore, many bioactive compounds elicit autoinduction of enzymes (e.g. in the hepatic system) that lead to increasing breakdown the drugs before they reach the systemic circulation, leading to a decrease of bioavailability of the molecules over time during a medicament administration regime. Despite these challenges, the oral route of drug administration remains the most common.

It is among the objectives of the present invention to attempt a solution to these problems.

### SUMMARY OF THE INVENTION

Accordingly, the invention provides a pharmaceutical composition for the sublingual delivery of a medicament comprising: a neutral oil; and a medicament soluble in said oil; wherein said medicament is in solution in said oil at a concentration providing a required dose in a volume of no more than 1 ml of composition; providing that said medicament is not nitroglycerine.

The requirement for a composition for sublingual drug delivery is very different than that for oral drug delivery. Oral drug delivery requires adsorption of the drug from the gastrointestinal tract for which the drug is ideally soluble in the aqueous solutions found there. However, for sublingual drug delivery the product needs to be lipophilic to be adsorbed from the sublingual region of the body. Thus, formulations having a hydrophilic nature of this patent would not result in good adsorption. Such formulations are at risk of being washed down into the gastrointestinal tract without being adsorbed. Many of the drugs that may be used for sublingual delivery in this way are not absorbed from the gastrointestinal tract, and might lead to undesirable side-effects.

Particular medicaments envisaged include especially opioids such as fentanyl and buprenorphine, pharmaceutically acceptable salts thereof, analogues thereof or derivatives thereof. Other opioids envisaged include: alfentanil, sufentanil, butorphanol, codeine, hydrocodone, hydromorphone, levorphanol, meperidine, methadone, morphine, nalbuphine, oxycodone, oxymorphbne, propoxyphene, tramadol, fenpipramide, piritramide, tilidine, tramadol, pharmaceutically acceptable salts thereof, or derivatives thereof, and the like.

Preferably, said medicament is in solution in said oil at a concentration providing a required dose of medicament in a volume of no more than 500 microlitres of composition; more preferably in a volume of no more than 200 microlitres of composition, and most preferably in a volume of no more than 100 microlitres of composition.

The use of such pharmaceutical compositions for delivery of medicaments by the sublingual route is appropriate, therefore, for those medicaments that have a suitably high solubility in neutral oils such that a required dose (e.g. an effective dose for a required pharmaceutical action) may be dissolved in a relatively small volume of composition, as above. This is particularly important, as the inventors have found that the sublingual delivery route offers (for many medicaments) substantial and hitherto unappreciated benefits over other administration routes. It is particularly beneficial over the oral route in which a medicament is often degraded by the various enzymatic and other processes in action in the gut, and leads to absorption by the hepatic route, which can lead to significant malabsorption as a result of the “first pass effect” in the liver. As a result, orally-dosed medicaments are often given in greater concentration that would be required if they were well-absorbed and could escape the first-pass effect. As a consequence, unwanted side-effects might be experienced. In order to avoid oral absorption, the medicament is therefore delivered in a small volume, enough to coat the sublingual mucosa and to reduce the likelihood that any composition may be swallowed. The skilled addressee will be readily able to determine whether a chosen medicament has sufficient solubility, and examples are given below to show how this might be done.

The invention is especially concerned with compositions for the delivery of medicaments by the sublingual route for systemic treatment of an individual, rather than for medicaments for use as a topical treatment.

A further preferred feature is that the medicament is stable in the composition, both with respect to physicochemical aspects such as remaining in solution and in terms of chemical (including biochemical) degradation of the medicament over time. It is particularly preferred, therefore that the medicament is stable within the composition, to pharmaceutically-acceptable limits over a period of at least one month, preferably 6 months and most preferably for a year.

Preferably, said neutral oil comprises a glyceride, and more preferably a triglyceride.

In especially preferred embodiments said triglyceride comprises miglyol, and especially a miglyol selected from the group comprising: miglyol 810; miglyol 812; miglyol 818; miglyol 829; and miglyol 840.

Also in especially preferred embodiments said neutral oil comprises an oil selected from the group comprising: Refined Maize Oil (Ph Eur); Virgin Castor Oil (Ph Eur); Refined Olive Oil (Ph Eur) and Refined Rapeseed Oil (Ph Eur).

Also in especially preferred embodiments said neutral oil comprises an oil selected from the group comprising: Glycerol mono-oleates (Ph Eur); Linoleoyl Macrogolglycerides (Ph Eur); Oleoyl Macrogolglycerides (Ph Eur); Vegetable Fatty Oils (Ph Eur); rich in triglycerides, Medium Chain Triglycerides (Ph Eur); Coconut Oil (Ph Eur); Fractionated Palm Kernel Oil (Ph Eur); Hydrogenated Cottonseed Oil (Ph Eur); Omega-3-Marine Triglycerides (Ph Eur); Fish Oil, Rich in Omega-3-Acids (Ph Eur); Cod Liver Oil (Ph Eur); Diglycerides; Monoglycerides; and Diglycerol.

Also in especially preferred embodiments said neutral oil comprises derivates or partial glycerides of an oil selected from the group comprising: Glycerol mono-oleates (Ph Eur); Linoleoyl Macrogolglycerides (Ph Eur); Oleoyl Macrogolglycerides (Ph Eur); Vegetable Fatty Oils (Ph Eur); rich in triglycerides, Medium Chain Triglycerides (Ph Eur); Coconut Oil (Ph Eur); Fractionated Palm Kernel Oil (Ph Eur); Hydrogenated Cottonseed Oil (Ph Eur); Omega-3-Marine Triglycerides (Ph Eur); Fish Oil, Rich in Omega-3-Acids (Ph Eur); Cod Liver Oil (Ph Eur); Diglycerides; Monoglycerides; and Diglycerol.

Medium chain length triglycerides are defined in the European Pharmacopoeia Monograph 0868, as:

A mixture of triglycerides of saturated fatty acids, mainly of caprylic acid (octanoic acid, C₈H₁₆O₂) and of capric acid (decanoic acid, C₁₀H₂₀O₂). Medium-chain triglycerides are obtained from the oil extracted from the hard, dried fraction of the endosperm of Cocos nucifera L. or from the dried endosperm of Elaeis guineensis Jacq. When Medium-chain Triglycerides are prepared from the endosperm of Cocos nucifera L., the title Fractionated Coconut Oil may be used. Medium chain length triglycerides have a minimum 95.0 percent of saturated fatty acids with 8 and 10 carbon atoms. Further chemical and physical properties are described in the European Pharmacopoeia Monograph 0868, and equivalent documents.

Omega-3-marine triglycerides are defined in the European Pharmacopoeia Monograph 0868 as mixture of mono-, di- and triesters of omega-3 acids with glycerol containing mainly triesters and obtained either by esterification of concentrated and purified omega-3 acids with glycerol or by transesterification of the omega-3 acid ethyl esters with glycerol. The origin of the omega-3 acids is the body oil from fatty fish species coming from families like Engraulidae, Carangidae, Clupeidae, Osmeridae, Salmonidae and Scombridae. The omega-3 acids are identified as the following acids: alpha-linolenic acid (C18:3 n-3), moroctic acid (C18:4 n-3), eicosatetraenoic acid (C20:4 n-3), timnodonic (eicosapentaenoic) acid (C20:5 n-3; EPA), heneicosapentaenoic acid (C21:5 n-3), clupanodonic acid (C22:5 n-3) and cervonic (docosahexaenoic) acid (C22:6 n-3; DHA). The sum of the contents of the omega-3 acids EPA and DHA, expressed as triglycerides is a minimum of 45.0 percent, and the total omega-3 acids, expressed as triglycerides is a minimum of 60.0 percent. Tocopherol may be added as an antioxidant.

Fish oil, rich in omega-3-acids is also defined in the European Pharmacopeia as purified, winterised and deodorised fatty oil obtained from fish of the families Engraulidae, Carangidae, Clupeidae, Osmeridae, Scombridae and Ammodytidae. The omega-3 acids are defined as the following acids: alpha-linolenic acid (C18:3 n-3), moroctic acid (C18:4 n-3), eicosatetraenoic acid (C20:4 n-3), timnodonic (eicosapentaenoic) acid (C20:5 n-3; EPA), heneicosapentaenoic acid (C21:5 n-3), clupanodonic acid (C22:5 n-3) and cervonic (docosahexaenoic) acid (C22:6 n-3; DHA).

The content of the Fish oil, rich in omega-3-acids is as follows:

EPA, expressed as triglycerides: minimum 13.0 percent,
DHA, expressed as triglycerides: minimum 9.0 percent,
Total omega-3-acids, expressed as triglycerides: minimum 28.0 percent.

In preferred embodiments any of said compositions, the compositions consist essentially of said neutral oil; and a medicament soluble in said oil.

In alternative embodiments of the above compositions, it is preferred that said composition further comprises a co-solvent selected from the group comprising: ethanol; isopropanol; propylene glycol; and polyethylene glycol.

In preferred embodiments any of said compositions, the compositions further comprise an excipient selected from the group comprising: an antioxidant; a preservative; a mucosal penetration enhancer, and a flavouring. Preferably, said flavouring or mucosal penetration enhancer comprises an essential oil such as menthol, vanillin or orange oil, lemon oil, clove oil, peppermint oil, spearmint oil. The inventors have found that the addition of such an essential oils surprisingly has three benefits: (1) the essential oils act as penetration enhancers, improving the rate and extent of uptake of such medicaments by the sublingual mucosa; (2) the essential oils, in many cases, act as co-solvents thereby increasing the solubility of medicaments; and (3) the essential oils provide a flavour component, giving organoleptic feedback to a user of the medicament, to confirm that is has been successfully delivered.

In preferred embodiments of any individual such composition, it is preferred that said medicament is not fentanyl, derivatives thereof such as sufentanil, carfentanil, lofentanil, alfentanil, or the like, and pharmaceutically acceptable salts thereof.

Also in preferred embodiments of any individual such composition, it is preferred that said medicament is not an artemesinin (including, without limitation, artemether, arteether and artesunate).

Also in preferred embodiments of any individual such composition, it is preferred that said medicament is not dihydropolyprenol (especially dihydroheptaprenol), probucol or tacrolimus.

Also in preferred embodiments of any individual such composition, it is preferred that said medicament is not a benzodiazepine.

In some conditions responsive to treatment with compositions or medicaments disclosed herein, patients may exhibit mucusitis and a dry mouth, especially when taking opioids. The inventors have found that miglyol may be used as the sole solvent for the active compounds (with the exception of buprenorphine, which requires the use of ethanol as a co-colvent); this allows formulations to exclude ethanol and other alcohols as a co-solvent, which is particularly beneficial, as alcoholic preparations are particularly irritating to a dry mouth, or to patients having mucusitis and may cause discomfort or pain to the patient. Accordingly, in preferred embodiments of compositions disclosed herein, the composition is substantially, or preferably entirely free of ethanol and more preferably substantially, or preferably entirely free of other alcohols. Formulations such as this have an additional benefit that they may be used in cultural or religious contexts where alcohol intake is not permitted.

Additionally, the additional of alcohols to such lipophilic compositions has the effect of reducing the particle size of droplets (by surface tension and viscosity effects) when the compositions are delivered in the form of a spray. This can lead to the formation of droplets less than 20 μm, or even less than 10 μm, which can allow droplets to reach the lungs, which is undesirable. Furthermore, alcohols can have the effect of “closing down” the mucosa, thereby having a deleterious effect on absorption of the medicament.

Also in embodiments of any individual such composition, it is preferred that said composition has less than 20% (w/w), more preferably less than 10% (w/w); more preferably still less than 5% (w/w); and most preferably less than 1% (w/w) of surfactant. In especially preferred embodiments, the composition is essentially free of surfactant. A key feature of the success of sublingual delivery is the provision of an essentially hydrophobic (lipophilic) composition; this leads to the composition remaining on the sublingual mucosa for absorption by that route. If surfactants are present within the composition, there is more likelihood that the composition will be able to mix with the essentially aqueous saliva in the mouth, leading to increased possibility that the composition will be moved away from the sublingual mucosa and, in extremis, swallowed by a user, thereby leading to oral rather than sublingual dosing.

Also included within the scope of the invention is a delivery device adapted to deliver successive doses of a composition according to any preceding claim, said doses comprising liquid droplets having a mean diameter of at least about 10 microns.

Preferably the compositions of the present invention are delivered as liquid droplets having a mean diameter of at least about 20 microns, more preferably a mean diameter of from about 20 to about 200 microns. Most preferably the formulations are delivered as liquid droplets have a size distribution of from about 5 microns to about 500 microns, preferably from about 10 microns to about 200 microns, preferably from about 20 microns to about 100 microns, more preferably from about 30 microns to about 70 microns. Choice of these droplet sizes ensures that the spray is prevented from passing into the lungs.

It is particularly preferred that each individual or successive dose has a volume of less than 1000 microlitres. The use of small dose volumes reduces the likelihood that the composition will be swallowed, or spat out, by the patient. The likelihood is reduced further by use of smaller volumes (especially in the paediatric context or for nasal delivery) and so in further preferred embodiments, each successive dose has a volume of less than 600 microlitres; less than 400 microlitres; less than 200 microlitres; or even less than 100 microlitres. Smaller volumes are especially preferred for paediatric use.

Preferably, the delivery devices according to these aspects comprise a spray, and especially a pump spray. The use of a pump spray increases the area of mucosa to which the composition is applied, thereby increasing absorption and minimising the likelihood that the medicament is swallowed.

### DESCRIPTION OF PREFERRED EMBODIMENTS

The inventors have found that the use of sublingual delivery of medicaments is more broadly useful in overcoming the problems of drug delivery described above than has hitherto been recognised. The sublingual venous bed drains into the systematic circulation rather than the hepatic circulation, and so the problems of the first pass effect are removed. Furthermore, the bypassing of the hepatic portal system during drug uptake prevents the autoinduction that, for many medicaments, leads to reduction of bioavailability of drugs on successive doses. The use of a sublingual delivery route also means that medicaments may be delivered, avoiding the oral route, by non-trained personnel, in contrast to the alternative of intravenous injection that might be used to avoid the first-pass effect. Additionally, some drugs are not able to be formulated for intravenous injection. Additional benefits of sublingual delivery are that, by careful choice of excipients and droplet sizes, accidental delivery of drug by the oral route can be avoided, thereby preventing the unwanted complications of the oral delivery route.

Whilst some sublingual formulations have been used, these are often formulated using propellants and irritant excipients such as alcohols. For some patients, e.g. those who might have sensitive mucosa as a symptom of their condition, these excipients are unwelcome. In some preferred embodiments, therefore, formulations specifically exclude propellants and alcoholic excipients.

By way of non-limiting example, the following formulations of oil-soluble medicaments are proposed:

### Example A

### Nicotine

| | % (w/w) | | | | | | |
|---|---|---|---|---|---|---|---|
| Nicotine | 1.06 | 1.06 | 1.06 | 1.06 | 1.06 | 1.06 | 1.06 |
| Clove oil | — | 1.06 | 1.07 | 1.06 | 1.06 | 1.06 | 1.06 |
| BHT* | — | — | 0.11 | 0.26 | 0.53 | 0.80 | 1.06 |
| Miglyol | 98.94 | 97.88 | 97.76 | 97.62 | 97.35 | 97.08 | 96.82 |

*Butylated hydroxy toluene

### Example B

### Buprenorphine

| | % (w/w) | | |
|---|---|---|---|
| Buprenorphine base | 1.1 | 4.4 | 8.4 |
| Ethanol abs. | 22.7 | 21.4 | 20.8 |
| Miglyol | 76.2 | 74.2 | 70.8 |

### Example C

### Fentanyl

| | % (w/w) | | | | | | | | | |
|---|---|---|---|---|---|---|---|---|---|---|
| Fentanyl Base | 0.06 | 0.06 | 0.06 | 0.06 | 0.06 | 0.23 | 0.23 | 0.23 | 0.23 | 0.23 |
| propyl parabens | — | — | 0.11 | 0.21 | 0.42 | — | — | 0.11 | 0.21 | 0.43 |
| Orange oil | — | 0.85 | 0.85 | 0.85 | 0.86 | — | 0.85 | 0.85 | 0.85 | 0.86 |
| miglyol | 99.94 | 99.09 | 98.96 | 98.99 | 98.66 | 99.77 | 89.92 | 98.81 | 98.71 | 98.48 |

Additional excipients found by the inventors to be readily soluble in miglyol, and therefore of us in formulation of the present invention include:

Flavourings: Orange oil; Lemon oil; Aniseed; Peppermint; and Menthol
Preservatives: Propyl parabens and Butyl parabens
Antioxidants: Butylated Hydroxy Toluene; Butylated Hydroxy Anisole and alpha tocopherol

It has been thought that oil-based excipients can lead to low absorption of medicaments. International Patent Application WO2007087431 teaches that “ . . . studies also showed that fentanyl base formulation containing Miglyol had very low permeability”. In contrast to these findings, the inventors have found that the use of oil-based excipients as recited herein, for oil-soluble drugs, surprisingly leads to highly efficient uptake of the medicaments.

As an example, the inventors have carried out confidential trials of sublingual uptake of the artemesinin arteether, described in co-pending International Patent Application PCT/GB2008/050999, and reproduced here:

Trials were carried out on healthy male adult human volunteers (16 subjects per cohort), and subject to normal ethical approval. Three single-dose regimes according to the present invention were studied, and compared to a regime using oral-dosed tablets, as follows:

### Sub-Lingual Spray Regimes

Spray formulations of artemether were prepared as detailed above, and administered, on a single occasion, to a group of volunteers by the sublingual route. A number of successive actuations of the spray were administered, as shown in Table 6, below.

TABLE 6
Dosage Regime for Single Dose Study
Sublingual Spray Formulation

| | | Dose per | Number of | Total Doge |
|---|---|---|---|---|
| Test | Formulation | Actuation (mg) | Actuations | (mg) |
| T1 | As Table 3 | 3 | 5 | 15 |
| T2 | As Table 3 | 3 | 10 | 30 |
| T3 | As Table 4 | 6 | 5 | 30 |

### Reference Oral Dose

As a reference, a fourth group of volunteers were administered tablets containing artemether, on a single occasion, as shown in Table 7, below.

TABLE 7
Dosage Regime for Single Dose Study
Oral Tablet Formulation

| | | Dose per Tablet | Number of | Total Doge |
|---|---|---|---|---|
| Test | Formulation | (mg) | Tablets | (mg) |
| T4 | Tablet | 10 | 3 | 30 |

Following administration of each dosage regime, blood samples were taken from the subjects, and plasma concentrations of artemether and its immediate metabolite dihydroartemesinin were determined, in order to compare bioavailability by the two routes.

FIGS. 1-6 show mean plasma concentration of artemether following two comparison dose regimes. FIGS. 7-12 show the corresponding mean plasma concentration of dihydroartemesinin.

FIGS. 1 and 7 compare regimes T1 (open squares) and T4 (closed circles): 15 mg artemether via 5 sublingual spray doses vs. 30 mg artemether via tablet.

FIGS. 2 and 8 compare regimes T2 (open squares) and T4 (closed circles): 30 mg artemether via 10 sublingual spray doses vs. 30 mg artemether via tablet.

FIGS. 3 and 9 compare regimes T3 (open squares) and T4 (closed circles): 30 mg artemether via 5 sublingual spray doses vs. 30 mg artemether via tablet.

FIGS. 4 and 10 compare regimes T1 (open squares) and T2 (closed circles): 15 mg artemether via 5 sublingual spray doses vs. 30 mg artemether via 10 sublingual spray doses.

FIGS. 5 and 11 compare regimes T2 (open squares) and T3 (closed circles): 30 mg artemether via 10 sublingual spray doses vs. 30 mg artemether via 5 sublingual spray doses.

FIGS. 6 and 12 compare regimes T1 (open squares) and T3 (closed circles): 15 mg artemether via 5 sublingual spray doses vs. 30 mg artemether via 5 sublingual spray doses).

Pharmacokinetic data for each of the four dosage regimes are given in Tables 8-11, below:

TABLE 8
Test Group T1
Single sublingual administration of 15 mg Artemether
sublingual spray: 3 mg per actuation

| | | Plasma |
|---|---|---|
| | Plasma Artemether | Dihydroartemesinin |
| Pharmacokinetic | (n = 16) | (n = 16) |
| Parameters* | (mean ± SD) | (mean ± SD) |
| AUC₀₋₁₂ (ng · h/mL) | 25.85 ± 13.88 | 29.63 ± 11.58 |
| Cₘₐₓ (ng/mL) | 16.11 ± 8.69 | 18.29 ± 7.52 |
| Tₘₐₓ (h) | 1.70 ± 0.68 | 1.83 ± 0.68 |
| t_{1/2} (h) | 0.72 ± 0.30 | |
| λ_{z} (h⁻¹) | 1.11 ± 0.40 | |
| CL/F (ng/h) | 0.74 ± 0.46 | 0.54 ± 0.15 |
| V/F (L) | 0.68 ± 0.33 | 0.51 ± 0.16 |

*Key:
AUC₀₋₁₂ (ng · h/mL) Area under the concentration curve between 0-12 h.
Cₘₐₓ (ng/mL) Maximum observed plasma concentration
Tₘₐₓ (h) Time of observed maximum plasma concentration
t_{1/2} (h) Elimination half-life
λ_{z} (h⁻¹) Elimination rate constant
CL/F (ng/h) Apparent clearance rate
V/F (L) Apparent volume of distribution

TABLE 9
Test Group T2
Single sublingual administration of 30 mg Artemether
sublingual spray: 3 mg per actuation

| | | Plasma |
|---|---|---|
| | Plasma Artemether | Dihydroartemesinin |
| Pharmacokinetic | (n = 16) | (n = 16) |
| Parameters | (mean ± SD) | (mean ± SD) |
| AUC₀₋₁₂ (ng · h/mL) | 76.60 ± 43.12 | 99.51 ± 50.33 |
| Cₘₐₓ (ng/mL) | 32.12 ± 16.39 | 44.11 ± 28.48 |
| Tₘₐₓ (h) | 1.73 ± 0.82 | 2.10 ± 1.17 |
| t_{1/2} (h) | 1.39 ± 0.49 | |
| λ_{z} (h⁻¹) | 0.56 ± 0.20 | |
| CL/F (ng/h) | 0.56 ± 0.37 | 0.36 ± 0.13 |
| V/F (L) | 1.00 ± 0.55 | 0.72 ± 0.36 |

Key as Table 8

TABLE 10
Test Group T3
Single sublingual administration of 30 mg Artemether
sublingual spray: 6 mg per actuation

| | | Plasma |
|---|---|---|
| | Plasma Artemether | Dihydroartemesinin |
| Pharmacokinetic | (n = 16) | (n = 16) |
| Parameters | (mean ± SD) | (mean ± SD) |
| AUC₀₋₁₂ (ng · h/mL) | 71.11 ± 41.08 | 86.19 ± 27.68 |
| Cₘₐₓ (ng/mL) | 35.24 ± 23.91 | 41.14 ± 16.45 |
| Tₘₐₓ (h) | 1.67 ± 0.77 | 1.88 ± 0.74 |
| t_{1/2} (h) | 1.40 ± 0.59 | |
| λ_{z} (h⁻¹) | 0.59 ± 0.25 | |
| CL/F (ng/h) | 0.63 ± 0.49 | 0.39 ± 0.15 |
| V/F (L) | 1.01 ± 0.49 | 0.91 ± 0.67 |

Key as Table 8

TABLE 11
Test Group T4
Single oral administration of 30 mg Artemether Tablets
10 mg per Tablet

| | | Plasma |
|---|---|---|
| | Plasma Artemether | Dihydroartemesinin |
| Pharmacokinetic | (n = 16) | (n = 16) |
| Parameters | (mean ± SD) | (mean ± SD) |
| AUC₀₋₁₂ (ng · h/mL) | 34.59 ± 21.01 | 38.49 ± 12.38 |
| Cₘₐₓ (ng/mL) | 10.12 ± 7.19 | 10.99 ± 4.39 |
| Tₘₐₓ (h) | 1.02 ± 0.86 | 1.39 ± 0.88 |
| t_{1/2} (h) | 3.44 ± 4.26 | |
| λ_{z} (h⁻¹) | 0.31 ± 0.15 | |
| CL/F (ng/h) | 1.11 ± 1.01 | 0.76 ± 0.23 |
| V/F (L) | 3.90 ± 2.90 | 2.36 ± 1.26 |

Key as Table 8

From these preliminary results, it can be seen that comparison of the area under the plasma concentration curve during the 12 hours following the doses (AUC₀₋₁₂), a well-accepted measure of absorption, shows significant and surprisingly higher absorption of artemether when administered sublingually as a spray formulation as disclosed herein by comparison to oral tablet dosing.

For comparison of bioavailability of artemether via the sublingual spray route described herein with administration by oral tablets, we have calculated the F-values, commonly used to compare two dose regimes, generally A and B, for the artemether data, as follows:

$${F}_{A - B} = \frac{{AUC}_{A}}{{AUC}_{B}} \frac{{dose}_{B}}{{dose}_{A}}$$

The results are as follows:

F_{T1-T4}=1.67±0.60 (S.D.)

F_{T2-T4}=2.24±0.92 (S.D.)

F_{T3-T4}=2.09±0.69 (S.D.)

This indicates that approximately between 1.7 and 2.2 times more artemether was absorbed when administered as a sublingual spray as described herein by comparison to oral administration by tablet, despite the oral dose being twice as large in the first instance. The indicative bioavailability by the sublingual route is therefore at least twice that by the oral route for equivalent doses.

Inspection of the data of Tables 8-11, and FIGS. 1-12 also confirms this general finding for the primary active metabolite of artemether (dihydroartemesinin)

### Avoidance of Autoinduction

It is known that both oral and rectal administration of artemesinins is associated with autoinduction of the drug metabolism in individuals (see e.g. Ashton M, Hai T N, Sy N D, Huong D X, Van Huong N, Nieu N T, Cong L D. “Artemisinin pharmacokinetics is time-dependent during repeated oral administration in healthy male adults.”, Drug Metab Dispos. 1998; 26:25-7, and “Retrospective analysis of artemisinin pharmacokinetics: application of a semiphysiological autoinduction model”, Asimus and Gordi, Br. J Clin Pharmacol. 2007 June; 63(6): 758-762). As a result, systemically circulating artemesinin declines with each successive dose, thereby reducing the effectiveness of drug dosage regimes.

In confidential trials, the inventors have found that administration of artemesinins by the transmucosal sublingual route avoids such autoinduction, leading to consistent uptake and accumulating systemic concentration of the active drug metabolite, dihydroartemesinin, thereby providing significant advantage in administration by the sublingual route. A similar avoidance of autoinduction is expected with delivery by the transmucosal buccal or nasal route.

In confidential trials, volunteers followed the following treatment: A single administration of 30 mg artemether sublingual spray 6 mg/actuation on days 1 and 5 following an overnight fast, and twice daily administrations of 30 mg artemether sublingual spray 3 mg/actuation on days 2, 3, and 4 following a morning or evening meal. Blood samples were collected for pharmacokinetic analysis at the following time points:

Day 1: Predose, 0.25, 0.5, 0.75, 1, 1.5, 2, 2.5, 3, 4, 6, 8, and 12 h after dosing. Days 2, 3, and 4: pre morning dose and 0.5, 1, 2 and 4 h after morning dose and pre evening dose and 1 hour after evening dose.

Day 5: Predose, 0.25, 0.5, 0.75, 1, 1.5, 2, 2.5, 3, 4, 6, 8, 12 h and 24 h after dosing. Pharmacokinetic analysis of plasma dihydroartemesinin on days 1 and 5 revealed an effectively identical response, indicating the lack of autoinduction. Plasma concentration curves are shown in FIG. 14.

### Solubility of Medicaments

By way of example, to show how the skilled addressee might determine whether such compositions are suitable for a given medicament, solubility tests have been carried out on a number of pharmaceutical actives as detailed below. All drugs were used at their lowest concentration as used in IV injections, with the exception of Amoxicillin and Diphenhydramine. Solutions were prepared in Miglyol 810.

Amoxicillin: 4 g of Amoxicillin was weighed into a beaker and 50 ml of Miglyol was added. This was then diluted to 100 ml with Miglyol. The pale yellow suspension was magnetically stirred but didn't dissolve. Amoxicillin appears not to be soluble in Miglyol. However, the Amoxicillin used contained other excipients.

Budesonide: 50 mg of Budesonide was weighed into a beaker and 50 ml of Miglyol was added. This was then diluted to 100 ml with Miglyol. After extensive magnetic stirring a suspension was seen that did not dissipate upon further dilution and subsequent stirring. After the addition of heat and menthol (to separate solutions) the Budesonide was seen to dissolve. Budesonide appears to be soluble with the addition of heat or menthol.

Diphenhydramine: 2.5 g of Diphenhydramine was weighed into a beaker and 50 ml of Miglyol was added. After stirring, a further 150 ml of Miglyol was added. A pale white cloudy suspension was seen that became less cloudy upon magnetic stirring. Diphenhydramine appears to be sparingly soluble in Miglyol.

Ketoprofen: 1 g of Ketoprofen was weighed into a beaker and 50 ml of Miglyol was added. A cloudy off-white suspension was seen that did not lighten upon magnetic stirring. Ketoprofen appears to be insoluble in Miglyol. (See below with respect to solubility enhancement.)

Ketorolac: 750 mg of Ketorolac was weighed into a beaker and 50 ml of Miglyol was added. After stirring, a further 50 ml of Miglyol was added. A Pale white, very cloudy suspension was seen that did not dissipate upon magnetic stirring. Ketorolac appears to be insoluble in Miglyol.

Lamivudine: 500 mg of Lamivudine was weighed into a beaker and 50 ml of Miglyol was added. After extensive magnetic stirring a cloudy white suspension was seen that did not dissipate. Lamivudine appears to be insoluble in Miglyol.

Lidocaine Base: 1.25 g of Lidocaine Base was weighed into a beaker and 50 ml of Miglyol was added. After magnetically stirring for approximately 15 minutes the solution became slightly less cloudy, and after a further 15 minutes stirring the solution became clear. Lidocaine Base is readily soluble in Miglyol.

Loratadine: 500 mg of Loratadine was weighed into a beaker and 50 ml of Miglyol was added. After magnetically stirring for 15 minutes a clear solution was observed. Loratadine is readily soluble in Miglyol.

Melatonin: 3.75 g of Melatonin was weighed into a beaker and 50 ml of Miglyol added. This was then further diluted to 100 ml then 200 ml with Miglyol. After magnetic stirring, a thick pale yellow suspension was seen. After initially diluting to 100 ml then to 200 ml the solution did not change. Melatonin appears to be insoluble in Miglyol.

Nalbuphine HCl: 500 mg of Nalbuphine HCl was weighed into a beaker and 50 ml of Miglyol was added. The suspension was magnetically stirred for approximately 40 minutes but no change was seen. Nalbuphine HCl is not soluble in Miglyol.

Naloxone: 100 mg of Naloxone was weighed into beaker and 50 ml of Miglyol was added. Upon magnetically stirring a cloudy solution was observed but no particulate matter was seen on the bottom. Naloxone appears to be sparingly soluble in Miglyol.

Naltrexone Base: 1 g of Naltrexone Base was weighed into a beaker and 50 ml of Miglyol was added. This was further diluted to 100 ml with Miglyol. For the first dilution a cloudy suspension was seen that did not dissipate. Upon the addition of 50 ml of Miglyol and further stirring the suspension appeared to lighten. Naltrexone Base appears to be sparingly soluble. It may dissolve completely at a lower concentration. (See below with respect to solubility enhancement.)

Ondansetron HCl: 1 g of Ondansetron HCl was weighed into a beaker and 50 ml of Miglyol was added. This was further diluted to 100 ml with Miglyol. A cloudy suspension was seen that did not dissolve upon magnetic stirring or the addition of 50 ml of Miglyol. Ondansetron HCl appears to be insoluble.

Prilocaine Base: 1.25 g of Prilocaine base was weighed into a beaker and 50 ml of Miglyol was added. Upon magnetically stirring for 5 minutes a clear solution was seen with slight particulate matter resting on the bottom that dissolved after standing. Prilocaine Base appears to be readily soluble in Miglyol.

Salbutamol Sulphate: 200 mg of Salbutamol Sulphate was weighed into a beaker and 50 ml of Miglyol was added. After extensive magnetic stirring a cloudy white suspension was seen. Salbutamol Sulphate appears to be insoluble in Miglyol.

Sildenafil Citrate: 1 g of Sildenafil Citrate was weighed into a beaker and 10 ml of Miglyol was added. This was further diluted to 50 ml with Miglyol. A dense white suspension was observed that did not dissipate upon magnetic stirring. Sildenafil Citrate appears to be insoluble in Miglyol.

Sildenafil Base: 1 g of Sildenafil Base was weighed into a beaker and 10 ml of Miglyol was added. This was further diluted to 50 ml with Miglyol. A dense white suspension was observed that did not dissipate upon magnetic stirring. Sildenafil Base appears to be insoluble in Miglyol.

Terbutaline Sulphate: 50 mg of Terbutaline Sulphate was weighed into a beaker and 50 ml of Miglyol was added. A fine suspension was seen that did not dissipate upon magnetic stirring. Terbutaline Sulphate appears to be insoluble in Miglyol.

Tramadol HCl: 2.5 g of Tramadol HCl was weighed into a beaker and 50 ml of Miglyol was added. A cloudy suspension was seen that did not dissipate upon magnetic stirring. Tramadol HCl appears to be insoluble in Miglyol.

Zidovudine: 500 mg of Zidovudine was weighed into a beaker and 50 ml of Miglyol was added. A cloudy white suspension was seen that did not dissipate upon stirring. Zidovudine appears to be insoluble in Miglyol.

### Solubility Enhancement by Essential Oils

Further tests established the solubility enhancement effect of heat and, surprisingly, the additional of an essential oil; menthol was used in this example.

Ketoprofen: 50 mg of Ketoprofen was weighed into a beaker and 50 ml of Miglyol was added. The samples dissolved with heat or menthol, thought much faster with heat. Ketoprofen is soluble in Miglyol with the addition of heat or menthol.

Naltrexone Base: 100 mg of Naltrexone Base was weighed into a beaker and 50 ml of Miglyol was added. The samples dissolved with heat or menthol, thought much faster with heat. Naltrexone Base appears to be soluble with the addition of heat or menthol.

For the medicaments tested above that showed good solubility in Miglyol (Lidocaine Base, Prilocaine Base, Loratadine and Budesonide), further studies were carried out to assess the solubility limits and to provide example formulations to guide the skilled addressee in applying the invention to formulation for other medicaments:

Lidocaine Base: An approximate solubility limit was found to be approximately 140 mg.ml⁻¹. Three formulations were made and are shown in Table 10.1. Prilocaine Base: An approximate solubility limit was found to be approximately 137 mg.ml⁻¹. Three formulations were made and are shown in Table 10.2. Loratadine: An approximate solubility limit was found to be approximately 20 mg.ml⁻¹. Three formulations were made and are shown in Table 10.3.

TABLE 10.1

| | Lidocaine | | | Final |
|---|---|---|---|---|
| | Base | | | Concentration of |
| Formulation | (g) | Menthol (g) | Miglyol (ml) | drug (mg · ml⁻¹) |
| 1 | 2.5053 | 0.600 | 100 | 25.1 |
| 2 | 5.0008 | 0.590 | 100 | 50.0 |
| 3 | 10.0152 | 0.605 | 100 | 100.2 |

TABLE 10.2

| | Prilocaine Base | | Final Concentration of |
|---|---|---|---|
| Formulation | (g) | Miglyol (ml) | drug (mg · ml⁻¹) |
| 1 | 2.5086 | 100 | 25.1 |
| 2 | 5.0111 | 100 | 50.0 |
| 3 | 10.0971 | 100 | 101.0 |

TABLE 10.3

| | | | Final Concentration |
|---|---|---|---|
| Formulation | Loratadine (g) | Miglyol (ml) | of drug (mg · ml⁻¹) |
| 1 | 1.0397 | 100 | 10.4 |
| 2 | 2.0176 | 100 | 20.2 |

### Further Formulation Work

Further work was undertaken on drugs thought previously insoluble in Miglyol in light of Budesonide appearing to be insoluble in Miglyol but upon further formulation dissolving with heat or menthol (see below). Example formulations are given below in Tables 10.4 and 10.5.

TABLE 10.4

| | | | | Final |
|---|---|---|---|---|
| | Ketoprofen | Menthol | | Concentration of |
| Formulation | (g) | (g) | Miglyol (ml) | drug (mg · ml⁻¹) |
| 1 | 0.0520 | 0.335 | 50 | 1.04 |
| 2 | 0.0500 | — | 50 | 1.00 |

TABLE 10.5

| | Naltrexone | | | Final |
|---|---|---|---|---|
| | Base | Menthol | | Concentration of |
| Formulation | (g) | (g) | Miglyol (ml) | drug (mg · ml⁻¹) |
| 1 | 0.1041 | 0.340 | 50 | 2.08 |
| 2 | 0.1061 | — | 50 | 2.12 |

Budesonide: A solubility limit was not established for this drug because it appeared not to be compatible with Miglyol. However, after using heat and menthol (separately) the Budesonide appeared to dissolve. Two formulations are shown in Table 10.6

TABLE 10.6

| | Budesonide | | Miglyol | Final Concentration |
|---|---|---|---|---|
| Formulation | (g) | Menthol (g) | (ml) | of drug (mg · ml⁻¹) |
| 1 | 0.0507 | 0.605 | 50 | 1.01 |
| 2 | 0.0503 | — | 50 | 1.01 |

These results demonstrate the ability of essential oils to act as solubilising agents.

### Stability of Medicaments

To assess the stability of example formulations, four of the medicaments (Lidocaine, Prilocaine, Laratadine and Budesonide) were filled into serum bottles, sealed and subjected to stability tests at a range of temperatures and relative humidity. The results are given in Tables 11.1 and 11.2.

TABLE 11.1

| | Stability | Time- | |
|---|---|---|---|
| Drug | Conditions | point | Observations |
| Lidocaine Base | 5° C., 25° C./60% | 24 Hours | No colour change, |
| (25 mg/ml) | RH, 30° C./65% RH | | and no apparent |
| | and 40° C./75% RH | | solubility issues |
| Lidocaine Base | 5° C., 25° C./60% | 24 Hours | No colour change, |
| (50 mg/ml) | RH, 30° C./65% RH | | and no apparent |
| | and 40° C./75% RH | | solubility issues |
| Lidocaine Base | 5° C., 25° C./60% | 24 Hours | No colour change, |
| (100 mg/ml) | RH, 30° C./65% RH | | and no apparent |
| | and 40° C./75% RH | | solubility issues |
| Prilocaine Base | 5° C., 25° C./60% | 24 Hours | No colour change, |
| (25 mg/ml) | RH, 30° C./65% RH | | and no apparent |
| | and 40° C./75% RH | | solubility issues |
| Prilocaine Base | 5° C., 25° C./60% | 24 Hours | No colour change, |
| (50 mg/ml) | RH, 30° C./65% RH | | and no apparent |
| | and 40° C./75% RH | | solubility issues |
| Prilocaine Base | 5° C., 25° C./60% | 24 Hours | No colour change, |
| (100 mg/ml) | RH, 30° C./65% RH | | and no apparent |
| | and 40° C./75% RH | | solubility issues |
| Loratadine | 5° C., 25° C./60% | 24 Hours | No colour change, |
| (10 mg/ml) | RH, 30° C./65% RH | | and no apparent |
| | and 40° C./75% RH | | solubility issues |
| Loratadine | 5° C., 25° C./60% | 24 Hours | No colour change, |
| (20 mg/ml) | RH, 30° C./65% RH | | and no apparent |
| | and 40° C./75% RH | | solubility issues |
| Budesonide + | 5° C., 30° C./65% RH | 24 Hours | No colour change, |
| Heat | and 40° C./75% RH | | and no apparent |
| (1 mg/ml) | | | solubility issues |
| Budesonide + | 5° C., 30° C./65% RH | 24 Hours | No colour change, |
| Menthol | and 40° C./75% RH | | and no apparent |
| (1 mg/ml) | | | solubility issues |

The samples were also checked at 4 days and 5 days. No colour change or solubility issues were apparent.

TABLE 11.2

| | Stability | Time- | |
|---|---|---|---|
| Drug | Conditions | point | Observations |
| Lidocaine Base | 5° C., 25° C./60% | 1 Month | No colour change, |
| (25 mg/ml) | RH, 30° C./65% RH | | and no apparent |
| | and 40° C./75% RH | | solubility issues |
| Lidocaine Base | 5° C., 25° C./60% | 1 Month | No colour change, |
| (50 mg/ml) | RH, 30° C./65% RH | | and no apparent |
| | and 40° C./75% RH | | solubility issues |
| Lidocaine Base | 5° C., 25° C./60% | 1 Month | No colour change, |
| (100 mg/ml) | RH, 30° C./65% RH | | and no apparent |
| | and 40° C./75% RH | | solubility issues |
| Prilocaine Base | 5° C., 25° C./60% | 1 Month | No colour change, |
| (25 mg/ml) | RH, 30° C./65% RH | | and no apparent |
| | and 40° C./75% RH | | solubility issues |
| Prilocaine Base | 5° C., 25° C./60% | 1 Month | No colour change, |
| (50 mg/ml) | RH, 30° C./65% RH | | and no apparent |
| | and 40° C./75% RH | | solubility issues |
| Prilocaine Base | 5° C., 25° C./60% | 1 Month | No colour change, |
| (100 mg/ml) | RH, 30° C./65% RH | | and no apparent |
| | and 40° C./75% RH | | solubility issues |
| Loratadine | 5° C., 25° C./60% | 1 Month | No colour change, |
| (10 mg/ml) | RH, 30° C./65% RH | | and no apparent |
| | and 40° C./75% RH | | solubility issues |
| Loratadine | 5° C., 25° C./60% | 1 Month | Small particulate |
| (20 mg/ml) | RH, 30° C./65% RH | | matter adhering to |
| | and 40° C./75% RH | | the bottom of the |
| | | | Serum bottle. No |
| | | | colour change. |
| Budesonide + | 5° C., 30° C./65% RH | 1 Month | No colour change, |
| Heat | and 40° C./75% RH | | and no apparent |
| (1 mg/ml) | | | solubility issues |
| Budesonide + | 5° C., 30° C./65% RH | 1 Month | No colour change, |
| Menthol | and 40° C./75% RH | | and no apparent |
| (1 mg/ml) | | | solubility issues |

Further stability tests were carried out with Ketoprofen and Naltrexone, and the results presented in Table 11.3.

TABLE 11.3

| | Stability | | |
|---|---|---|---|
| Drug | Conditions | Time-point | Observations |
| Ketoprofen | 5° C., 30° C./65% | 24 Hours | No colour change, |
| (1 mg/ml) + | RH and 40° C./75% | | and no apparent |
| Menthol | RH | | solubility issues |
| Ketoprofen | 5° C., 30° C./65% | 24 Hours | No colour change, |
| (1 mg/ml) + | RH and 40° C./75% | | and no apparent |
| Heat | RH | | solubility issues |
| Naltrexone | 5° C., 30° C./65% | 24 Hours | No colour change, |
| Base | RH and 40° C./75% | | and no apparent |
| (2 mg/ml) + | RH | | solubility issues |
| Menthol | | | |
| Naltrexone | 5° C., 30° C./65% | 24 Hours | No colour change, |
| Base | RH and 40° C./75% | | and no apparent |
| (2 mg/ml) + | RH | | solubility issues |
| Heat | | | |

These samples were also checked after 5 and 6 days and no colour change or solubility issues were noted.

### SUPPLEMENTARY FIGURE CAPTIONS

FIG. 1: Plot of mean plasma Artemether concentration vs time with standard deviation following a single sublingual administration of 15 mg Artemether Sublingual Spray 3 mg/actuation (T1) and single oral administration of 30 mg Artemether Tablets 10 mg/tablet (T4). Mean±SD (•=reference, T4, □=test, T1)

FIG. 2: Plot of mean plasma Artemether concentration vs time with standard deviation following a single sublingual administration of 30 mg Artemether Sublingual Spray 3 mg/actuation (T2) and single oral administration of 30 mg Artemether Tablets 10 mg/tablet (T4). Mean±SD (•=reference, T4, □=test, T2)

FIG. 3: Plot of mean plasma Artemether concentration vs time with standard deviation following a single sublingual administration of 30 mg Artemether Sublingual Spray 6 mg/actuation (T3) versus single oral administration of 30 mg Artemether Tablets 10 mg/tablet (T4). Mean±SD (•=reference, T4, □=test, T3)

FIG. 4: Plot of mean plasma artemether concentration vs time with standard deviation following a single sublingual administration of 15 mg Artemether Sublingual Spray 3 mg/actuation (T1) versus single sublingual administration of 30 mg Artemether Sublingual Spray 3 mg/actuation (T2). Mean±SD (•=reference, T2, □=test, T1)

FIG. 5: Plot of mean plasma Artemether concentration vs time with standard deviation following a single sublingual administration of 30 mg Artemether Sublingual Spray 3 mg/actuation (T2) versus single sublingual administration of 30 mg Artemether Sublingual Spray 6 mg/actuation (T3). Mean±SD (•=reference, T3, □=test, T2)

FIG. 6: Plot of mean plasma Artemether concentration vs time with standard deviation following a single sublingual administration of 15 mg Artemether Sublingual Spray 3 mg/actuation (T1) versus single sublingual administration of 30 mg Artemether Sublingual Spray 6 mg/actuation (T3). Mean±SD (•=reference, T3, □=test, T1)

FIG. 7: Plot of mean plasma Dihydroartemisinin concentration vs time with standard deviation following a single sublingual administration of 15 mg Artemether Sublingual Spray 3 mg/actuation (T1) and single oral administration of 30 mg Artemether Tablets 10 mg/tablet (T4). Mean±SD (•=reference, T4, □=test, T1)

FIG. 8: Plot of mean plasma Dihydroartemisinin concentration vs time with standard deviation following a single sublingual administration of 30 mg Artemether Sublingual Spray 3 mg/actuation (T2) and single oral administration of 30 mg Artemether Tablets 10 mg/tablet (T4). Mean±SD (•=reference, T4, □=test, T2)

FIG. 9: Plot of mean plasma Dihydroartemisinin concentration vs time with standard deviation following a single sublingual administration of 30 mg Artemether Sublingual Spray 6 mg/actuation (T3) versus single oral administration of 30 mg Artemether Tablets 10 mg/tablet (T4). Mean±SD (•=reference, T4, □=test, T3)

FIG. 10: Plot of mean plasma Dihydroartemisinin concentration vs time with standard deviation following a single sublingual administration of 15 mg Artemether Sublingual Spray 3 mg/actuation (T1) versus single sublingual administration of 30 mg Artemether Sublingual Spray 3 mg/actuation (T2). Mean±SD (•=reference, T2, □=test, T1)

FIG. 11: Plot of mean plasma Dihydroartemisinin concentration vs time with standard deviation following a single sublingual administration of 30 mg Artemether Sublingual Spray 3 mg/actuation (T2) versus single sublingual administration of 30 mg Artemether Sublingual Spray 6 mg/actuation (T3). Mean±SD (•=reference, T3, □=test, T2)

FIG. 12: Plot of mean plasma Dihydroartemisinin concentration vs time with standard deviation following a single sublingual administration of 15 mg Artemether Sublingual Spray 3 mg/actuation (T1) versus single sublingual administration of 30 mg Artemether Sublingual Spray 6 mg/actuation (T3). Mean±SD (•=reference, T3, □=test, T1)

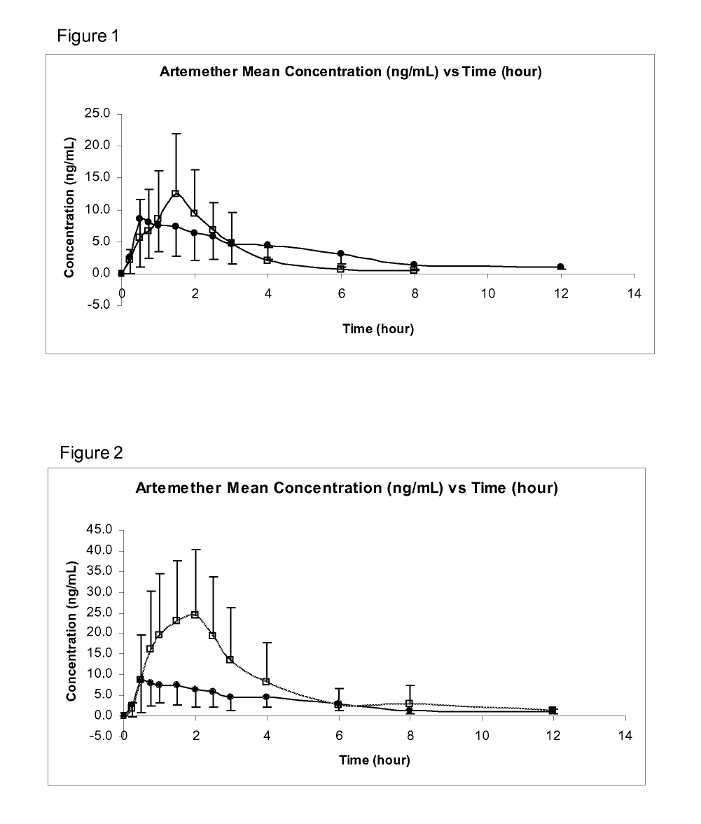

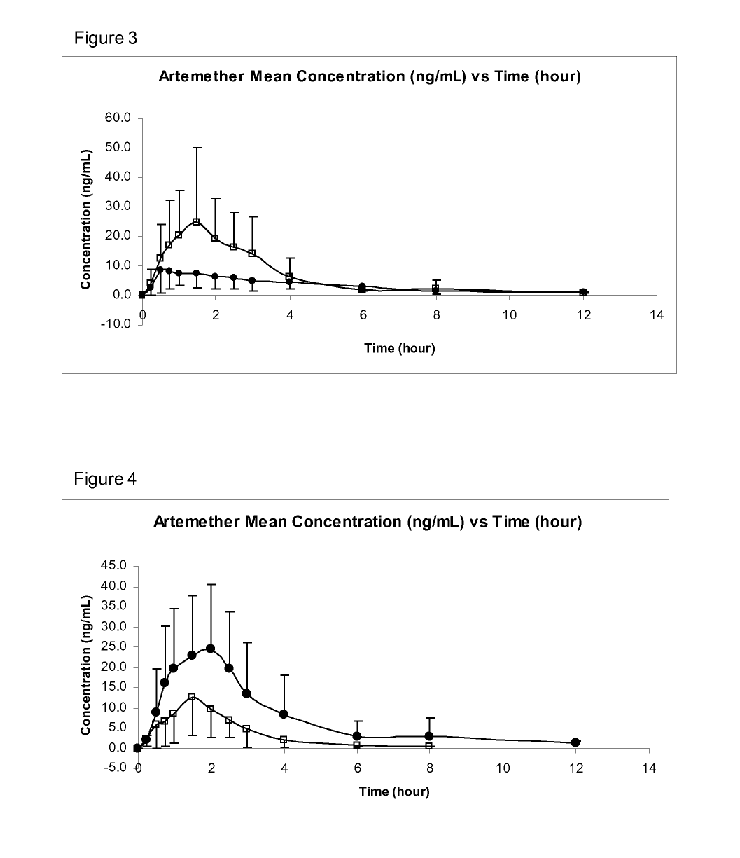

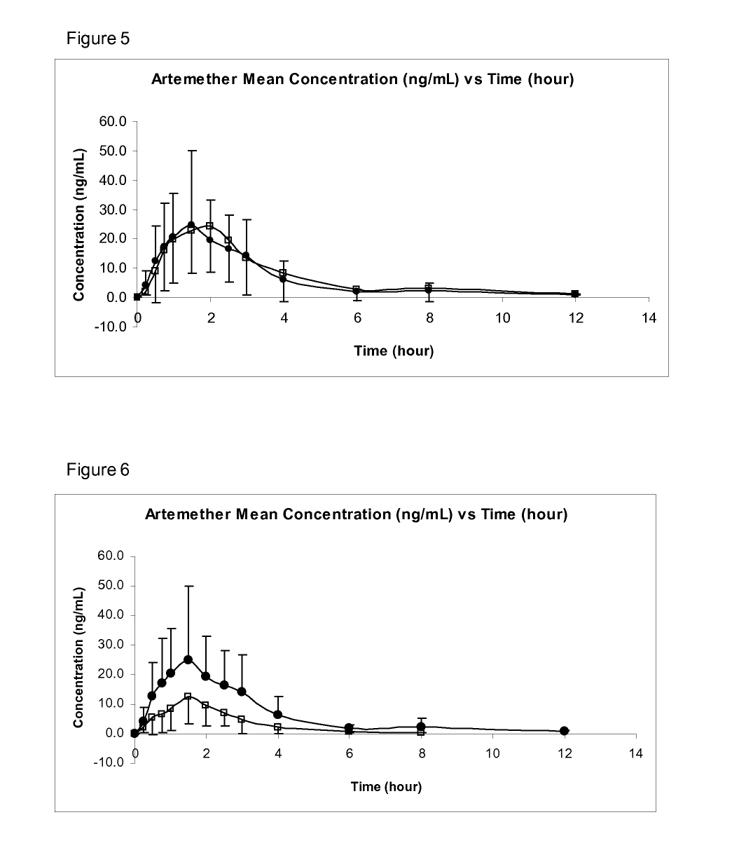

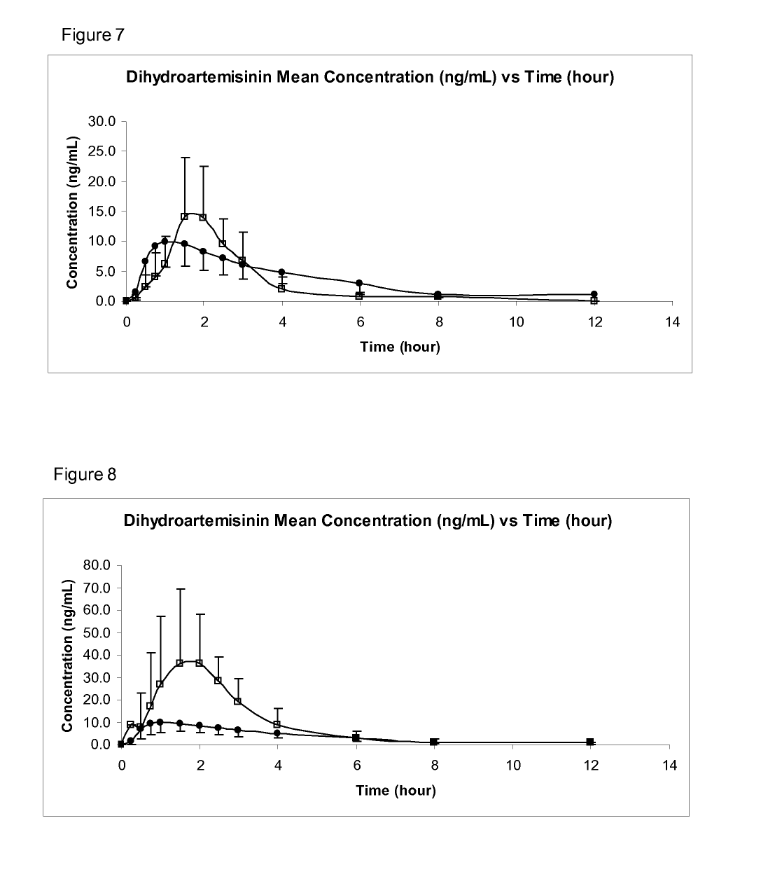

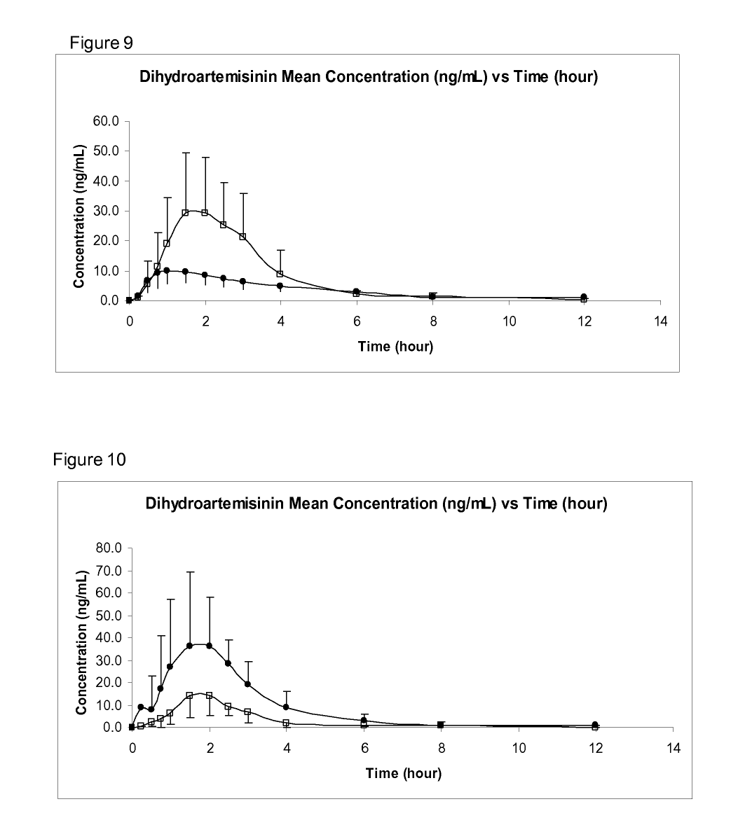

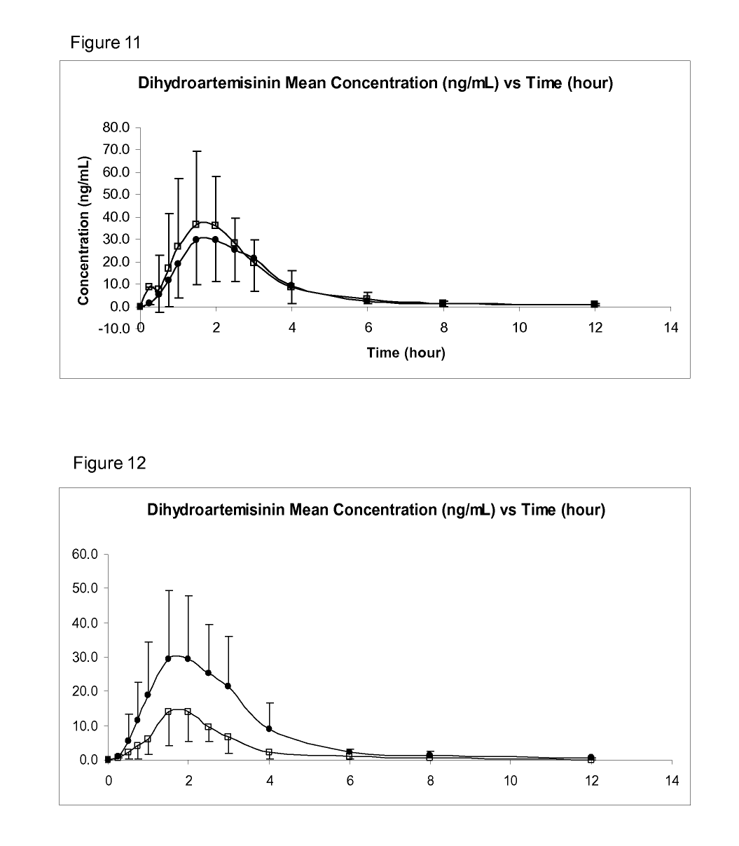

1. A pharmaceutical composition for use in the sublingual delivery of a medicament to a human, said composition comprising:
a neutral oil; and
a medicament soluble in said oil;
wherein said medicament is in solution in said oil at a concentration providing a required dose in a volume of no more than 1 ml of composition;
providing that said medicament is not nitroglycerine.

2. A composition according to claim 1 providing that said medicament is not fentanyl, derivatives thereof such as sufentanil, carfentanil, lofentanil, alfentanil, or the like, and pharmaceutically acceptable salts thereof.

3. A composition according to claim 1 providing that said medicament is not an artemesinin (including, without limitation, artemether, arteether and artesunate).

4. A composition according to claim 1 providing that said medicament is not dihydropolyprenol (especially dihydroheptaprenol), probucol or tacrolimus.

5. A pharmaceutical composition for the sublingual delivery of a medicament, said composition comprising:
a neutral oil; and
an opioid medicament soluble in said oil;
wherein said opioid medicament is in solution in said oil at a concentration providing a required dose in a volume of no more than 1 ml of composition;
providing that said opioid is not fentanyl, derivatives thereof such as sufentanil, carfentanil, lofentanil, alfentanil, or the like, and pharmaceutically acceptable salts thereof.

6. A composition according to claim 5 wherein said neutral oil comprises a glyceride.

7. A composition according to claim 6 wherein said glyceride comprises a triglyceride.

8. A composition according to claim 7 wherein said triglyceride comprises miglyol.

9. A composition according to claim 8 wherein said miglyol comprises miglyol selected from the group consisting of:
miglyol 810;
miglyol 812;
miglyol 818;
miglyol 829; and
miglyol 840.

10. A composition according to any claim 1 wherein said neutral oil comprises an oil selected from the group consisting of:
Refined Maize Oil (Ph Eur);
Virgin Castor Oil (Ph Eur);
Refined Olive Oil (Ph Eur) and
Refined Rapeseed Oil (Ph Eur).

11. A composition according to claim 1 wherein said neutral oil comprises an oil selected from the group consisting of:
Glycerol mono-oleates (Ph Eur);
Linoleoyl Macrogolglycerides (Ph Eur);
Oleoyl Macrogolglycerides (Ph Eur);
Vegetable Fatty Oils (Ph Eur); rich in triglycerides
Medium Chain Triglycerides (Ph Eur);
Coconut Oil (Ph Eur);
Fractionated Palm Kernel Oil (Ph Eur);
Hydrogenated Cottonseed Oil (Ph Eur);
Omega-3-Marine Triglycerides (Ph Eur);
Fish Oil, Rich in Omega-3-Acids (Ph Eur);
Cod Liver Oil (Ph Eur);
Diglycerides;
Monoglycerides;
Diglycerol.

12. A composition according to claim 1 wherein said neutral oil comprises derivates or partial glycerides of an oil selected from the group consisting of:
Glycerol mono-oleates (Ph Eur);
Linoleoyl Macrogolglycerides (Ph Eur);
Oleoyl Macrogolglycerides (Ph Eur);
Vegetable Fatty Oils (Ph Eur); rich in triglycerides
Medium Chain Triglycerides (Ph Eur);
Coconut Oil (Ph Eur);
Fractionated Palm Kernel Oil (Ph Eur);
Hydrogenated Cottonseed Oil (Ph Eur);
Omega-3-Marine Triglycerides (Ph Eur);
Fish Oil, Rich in Omega-3-Acids (Ph Eur);
Cod Liver Oil (Ph Eur);
Diglycerides;
Monoglycerides;
Diglycerol.

13. A composition according to claim 1 consisting essentially of said neutral oil and said medicament.

14. A composition according to claim 1, substantially free of ethanol.

15. A composition according to claim 14, substantially free of alcohols.

16. A composition according to claim 1, further comprising a co-solvent selected from the group consisting of:
ethanol;
isopropanol;
propylene glycol;
polyethylene glycol.

17. A composition according to claim 1, further comprising an excipient selected from the group consisting of:
an antioxidant;
a preservative;
a mucosal penetration enhancer;
a flavouring.

18. A composition according to claim 17 wherein said mucosal penetration enhancer comprises an essential oil.

19. A composition according to claim 17 wherein said flavouring comprises an essential oil.

20. A composition according to claim 1 having less than 20% (w/w) of surfactant.

21. A composition according to claim 20 having less than 10% (w/w) of surfactant.

22. A composition according to claim 21 having less than 5% (w/w) of surfactant.

23. A composition according to claim 22 having less than 1% (w/w) of surfactant.

24. A composition according to claim 23 essentially free of surfactant.

25. A composition according to claim 1 that is comprised within a delivery device adapted to deliver successive doses of said composition, said doses comprising liquid droplets having a mean diameter of at least about 10 microns.

26. A composition according to claim 25 wherein said droplets have a mean diameter of at least about 20 microns.

27. A composition according to claim 25 wherein said droplets have a mean diameter of from about 20 to about 200 microns.

28. A composition according to claim 25 wherein said doses are delivered by a pump spray.
